(19) United States
(12) Patent Application Publication
(10) Pub. No.: US 2003/0161885 A1
(43) Pub. Date: Aug. 28, 2003

(54) Agent having prolonged stomach retention time used to produce a longlasting saturation effect, and the use thereof

(76) Inventors: Gunther Beisel, Monheim (DE); Rudiger Groning, Munster (DE)

Correspondence Address: Friedrick Kueffner, Suite 910, 317 Madison Avenue, New York, NY, 10017, US

(21) Appl. No.: 10/363,090

(22) PCT Filed: Sep. 7, 2001
(86) PCT No.: PCT/EP01/10309

(30) Foreign Application Priority Data
Sep. 11, 2000 (DE) 10044846.1

(51) Int. Cl. A61K 31/715; A61K 9/14

(52) U.S. Cl. 424/486; 514/54

(57) ABSTRACT

The present invention relates to a composition for oral intake to generate a long-lasting satiation effect, and its use as additive to food products, for producing food supplements, appetite suppressants and/or satiating agents.

The present invention relates to a composition for oral intake to generate a long-lasting satiation effect, comprising at least one substance which increases the viscosity of a liquid, and at least one other compound which increases the retention time of the viscosity-increasing substance in the stomach.

Preparations with gel-forming substances which, in the form of tablets, granules, suspensions or solutions, display their long-lasting, satiating effect via the gastrointestinal tract are employed for the treatment for example of obesity and disorders caused by obesity.

Corresponding preparations comprise vegetable mucilages and swelling agents such as, for example, alginates, pectin, wheat bran, starch gels or guar gum. Together with an ingested liquid or in the presence of gastric fluid, these substances form a gelatinous structure, called hydroyds or hydrocolloids, which may serve inter alia for satiation.

The problem with the gelatinous compositions in general is that they are transported comparatively quickly through the exit from the stomach (pylorus) further into the small intestine and cause only brief satiation. The retention time of conventional preparations is, despite the high viscosity which they develop in the stomach, only slightly longer than the retention time of liquids. It is disadvantageous that the gelatinous compositions cannot, because of their rapid passage through the stomach, be employed for long-lasting filling of the stomach and achievement of a long-lasting satiation effect.

It is therefore desirable to provide gelatinous or semisolid compositions which have a longer stomach retention time and, associated with this long-lasting filling of the stomach, thus exert a long-term satiation effect.

This object is achieved in an advantageous manner by the present invention.

The present invention relates to a composition for oral intake to generate a long-lasting satiation effect, comprising at least one substance which increases the viscosity of a liquid, and at least one other compound which increases the retention time of the viscosity-increasing substance to prolong a satiation effect in the stomach.

Fatty acids and/or fatty acid derivatives are added according to the invention as further compound to the substances used for gel or puree formation and for increasing the viscosity of liquids. Fatty acids and/or fatty acid derivatives lead to a slowing of gastric emptying and thus to the desired increased stomach retention time. It may be mentioned in this connection that the compositions of the invention contain no medicinal substances having a systemic pharmacological effect. Medicinal substances having a systemic pharmacological effect mean those medicinal substances which, after absorption, are distributed in the blood circulation and reach the site of action by this route.

The compositions of the invention are further distinguished by comprising fatty acids having at least six carbon atoms in the molecule, their salts and/or derivatives thereof. These may be according to the invention saturated and/or unsaturated fatty acids.

In implementation variants of the present invention the compositions of the invention may comprise fatty acids having a carbon chain with a length in the range from C₁₀ to C₂₀, preferably C₁₂ to C₁₈, particularly preferably C13 to C16. The use of edible fatty acids is mentioned here by way of example, but these are not limiting for the present invention.

Salts and/or derivatives of the fatty acids are also encompassed according to the invention. These may be, for example, alkali metal and/or ammonium salts and/or various fatty acid esters. Examples thereof are glycerol esters such as, for example, fats in which the fatty acids may be present in bound form. The fatty acids may also be in chemically bound form in lecithin and be liberated by enzymatic processes during digestion in the gastrointestinal tract in order subsequently to bring about the desired function of prolonging the retention time of the composition of the invention.

The fatty acids can be incorporated as fine dispersions in the composition of the invention by trituration with colloidal silica. In the form of alkali metal salts and/or ammonium compounds, the solubility of the fatty acids in the composition of the invention is improved. It is possible in this case for the composition of the invention to comprise fatty acids in an amount of from 0.7 to 70 mg/g, preferably 2.5 to 50 mg/g and particularly preferably 10 to 20 mg/g of viscosity-increasing substance.

A viscosity-increasing substance means according to the invention a usually organic and high molecular weight substance which is able to take up liquids and thereby swell. Other designations are also thickener, swelling agent, gel former or hydrocolloid. These substances are converted, on uptake of liquid, into a viscous true or colloidal solution and then form gels or mucilages. Gels mean according to the invention dimensionally stable, easily deformable systems which are produced through the interaction of high molecular weight substances with liquids. The substances referred to as gel formers form three-dimensional networks in which liquid molecules (disperse medium) are incorporated. If the dispersion medium is a liquid, the term used is lyogels, and if it is water the gels are called hydrogels.

The gels do not, however, have firm connections between the cavities filled with liquid or gas as is the case, for example, in sponges or sponge-like structures.

The composition for achieving a long-lasting satiation effect comprises according to the invention besides the aforementioned fatty acids, their salts and/or derivatives as viscosity-increasing substance natural, synthetic and/or semisynthetic polysaccharides and/or polyacids and/or mixtures thereof.

Numerous examples thereof are known, such as, for example, agar-agar, alginic acid, alginates, laminarin, fucoidin, lentinan, schitophyllan, gum acacia, carrageenan, guar gum, locust bean gum, galactomannans, tragacanth, hyaluronic acid, althea mucilage, quince mucilage, fleaseed mucilage, xanthan, methylcellulose, carboxymethylcellulose, pectins, dextrans, dextrin, cellulose, gum arabic, gelatin, soya, starch and/or their derivatives and/or mixtures thereof.

Preferred embodiments of the composition of the invention comprise guar gum and/or methylcellulose and/or starch derivatives. Particularly preferred according to the invention are guar gallactomannan and/or guaran and/or hydroxymethylcellulose, hydroxymethylpropylcellulose, hydroxyethylcellulose, carboxymethylcellulose and/or hydroxyethylstarch and/or mixtures of the aforementioned substances.

The composition is in the form according to the invention of a powder, granulation, compact, pellet, tablet, capsule, solution, dispersion, puree and/or any other suitable formulation.

In one implementation variant of the present invention, the composition can be dissolved and/or dispersed in a hydrophilic or lipophilic medium. Media which are particularly suitable according to the invention are water, milk drinks, fruit juices and/or oil. It is possible in this case for the preparation to take place by stirring the composition of the invention into these liquids and/or be processed to a puree by heating.

The composition of the invention may further contain gas-forming substances. These gas-forming substances may on the one hand assist the formation of a gel. In a special variant, the composition of the invention may comprise effervescent mixtures. The effervescent mixture can preferably be with taste.

The present invention also relates to the use of an aforementioned composition as additive to food products, for producing food supplements, appetite suppressants and/or satiating agents.

A further possibility is for the composition of the invention not to be used as additives to food products but also to be taken before, during and/or after the consumption of food products and thus to bring about a longer retention time of the food product associated with a long-lasting satiation effect in the stomach.

The present invention is explained in more detail below by exemplary embodiments which, however, have no limiting effect on the invention:

### PREPARATION EXAMPLE 1

| Formula: | |
|---|---|
| Guar gum | 100 parts |
| C₁₄ fatty acid | 3.3 parts |
| Colloidal silica | 0.2 parts |

The C₁₄ fatty acid is finely triturated with the colloidal silica and then screened (mesh width 0.3 mm). The trituration of the fatty acid with the colloidal silica is mixed with the guar gum. This is followed by agglomerating granulation with purified water. The granules are dried at 45° C.

### PREPARATION EXAMPLE 2

| Formula: | |
|---|---|
| Hydroxypropylcellulose | 100 parts |
| Edible fatty acids | 3.3 parts |

5 parts of hydroxypropylcellulose are triturated with 3.3 parts of edible fatty acids. The trituration is homogeneously mixed with the remaining hydroxypropylcellulose.

### PREPARATION EXAMPLE 3

| Formula: | |
|---|---|
| Wheat bran | 100 parts |
| Isopropyl myristate | 5 parts |

5 parts of isopropyl myristate are triturated with 10 parts of wheat bran. The trituration is homogeneously mixed with the remaining wheat bran.

### PREPARATION EXAMPLE 4

| Formula: | |
|---|---|
| Edible fatty acid | 5 parts |
| Sodium chloride | 75 parts |
| Colloidal silica | 1 part |

The edible fatty acids are homogeneously triturated with the sodium chloride and the colloidal silica.

### USE EXAMPLE 1

3 to 6 g of the granules described in preparation example 1 are dispersed by stirring in 150 ml of multivitamin juice. The suspension of the granules is drunk within the first minute after addition to the multivitamin juice. A feeling of satiation lasting about 2 to 3 hours results.

### USE EXAMPLE 2

10 g of the mixture described in preparation example 3 are dispersed and processed to a puree in 150 g of fruit juice with heating. Consumption of the semiliquid preparation results in a feeling of satiation lasting 2 to 3 hours.

### USE EXAMPLE 3

4 to 8 g of the mixture described in preparation example 2 are stirred into food (soups, yogurts, purees) and eaten. The stomach retention time of the food is increased after consumption. The satiation effect is considerably prolonged thereby.

### USE EXAMPLE 4

2 to 10 g of the formula described in preparation example 4 are added to baking mixtures. The baked food product remains longer in the stomach after consumption than does the food product without added composition of the invention of formula 4.

1. A composition for oral intake to generate a long-lasting satiation effect, comprising at least one substance which increases the viscosity of a liquid, and at least one other compound which increases the retention time of the viscosity-increasing substance to prolong a satiation effect in the stomach.

2. The composition as claimed in claim 1, characterized in that it comprises as compound increasing the stomach retention time fatty acids and/or fatty acid derivatives which contribute to prolonging the satiation phase.

3. The composition as claimed in either of claims 1 or 2, characterized in that it comprises fatty acids having at least 6 carbon atoms in the molecule, their salts and/or derivatives thereof.

4. The composition as claimed in any of claims 1 to 3, characterized in that it comprises fatty acids having a carbon chain with a length in the range from C₁₀ to C₂₀, preferably from C₁₂ to C₁₈ and particularly preferably from C₁₃ to C_{16.}

5. The composition as claimed in any of claims 1 to 4, characterized in that it comprises saturated and/or unsaturated fatty acids and/or their derivatives.

6. The composition as claimed in any of claims 1 to 5, comprising fatty acids in an amount of from 0.7 to 70 mg/g, preferably 2.5 to 50 mg/g and particularly preferably 10 to 20 mg/g of viscosity-increasing substance.

7. The composition as claimed in any of claims 1 to 6, characterized in that it comprises as viscosity-increasing substance natural, synthetic, semisynthetic polysaccharides and/or polyacids and/or mixtures thereof.

8. The composition as claimed in any of claims 1 to 7, characterized in that it is in the form of a powder, granulation, compact, pellet, tablet, capsule, solution, dispersion and/or puree.

9. The composition as claimed in any of claims 1 to 8, characterized in that it is dissolved and/or dispersed in a hydrophilic or lipophilic medium.

10. The composition as claimed in any of claims 1 to 9, characterized in that it comprises gas-forming substances.

11. The composition as claimed in any of claims 1 to 10, characterized in that it comprises effervescent mixtures with taste.

12. The use of a composition as claimed in any of claims 1 to 11 as additive to food products, for producing food supplements, appetite suppressants and/or satiating agents.
